(19) United States
(12) Patent Application Publication
(10) Pub. No.: US 2021/0001050 A1
(43) Pub. Date: Jan. 7, 2021

(54) SYRINGE ASSEMBLY AND PREFILLED SYRINGE

(71) Applicant: TERUMO KABUSHIKI KAISHA, Tokyo (JP)

(72) Inventors: Manabu ARINOBE, Kanagawa (JP); Yusuke HYAKKAN, Kanagawa (JP); Eri OYA, Kanagawa (JP); Taeko MASUDA, Kanagawa (JP)

(73) Assignee: TERUMO KABUSHIKI KAISHA, Tokyo (JP)

(21) Appl. No.: 17/025,005

(22) Filed: Sep. 18, 2020

Related U.S. Application Data
(63) Continuation of application No. PCT/JP2019/011870, filed on Mar. 20, 2019.

(30) Foreign Application Priority Data
Mar. 20, 2018 (JP) 2018-051898

(51) Int. Cl. A61M 5/31; A61M 5/32; A61M 5/142

(52) U.S. Cl. CPC A61M 5/31; A61M 5/3202; A61M 5/3129; A61M 2205/8206; A61M 2005/3131; A61M 2005/3104; A61M 5/14248

(57) ABSTRACT

A syringe assembly has a body section including a lumen fillable with 0.3 to 1 mL of drug solution. The total length of the syringe assembly is 30 to 40 mm. The body section has an inner diameter of 8.0 to 9.0 mm and an outer diameter of 10 to 12 mm. The ratio of the outer diameter of the body section and the total length of the syringe assembly is 1:2.5 to 1:4. A flange portion includes two major diameter forming portions that are symmetrically provided with an axis of the body section interposed therebetween to form a major diameter of the syringe assembly. A minor diameter of the syringe assembly is 10 to 13 mm and is equal to or larger than the outer diameter of the body section. The major diameter is 12 to 15 mm and is larger than the minor diameter.

### CROSS-REFERENCES TO RELATED APPLICATIONS

This application is a continuation of International Application No. PCT/JP2019/011870 filed on Mar. 20, 2019, which claims priority to Japanese Patent Application No. 2018-051898 filed on Mar. 20, 2018, the entire content of each of which is incorporated herein by reference.

### FIELD OF THE INVENTION

The present invention generally relates to a syringe assembly and a prefilled syringe.

### BACKGROUND DISCUSSION

Known syringe pump-type drug solution administration devices, such as disclosed in WO 2013/148270 A, deliver a drug solution, with which a cylinder of the device is filled, into a living body by a pressing operation of a plunger.

### SUMMARY

In recent years, progress has been made in reducing the size of such drug solution administration devices. Furthermore, a type to be attached to the body has also appeared. In particular, in a drug solution administration device of the type that is to be attached to the body, it is important to reduce the size of the drug solution administration device so as to be as small as possible, from the viewpoint of limiting the size of the attachable area, as well as general usability.

A syringe assembly and a prefilled syringe disclosed here can more effectively reduce the size of a drug solution administration device.

A syringe assembly according to embodiments disclosed here includes: a body section including a lumen that is fillable with 0.3 to 1 mL of a drug solution; a distal nozzle portion that is provided at a distal end of the body section and has a distal opening communicating with the lumen of the body section; and a proximal portion that is provided at a proximal end of the body section. A total length of the syringe assembly is 30 to 40 mm. The body section has an inner diameter of 8.0 to 9.0 mm and an outer diameter of 10 to 12 mm. A ratio of the outer diameter of the body section and the total length of the syringe assembly is 1:2.5 to 1:4. The proximal portion includes a proximal opening that communicates with the lumen of the body section, a proximal surface that encircles the proximal opening, is orthogonal to an axis of the body section, and is substantially flat, and a flange portion that protrudes outward from an outer peripheral surface of the body section such that the syringe assembly has a minor diameter and a major diameter in directions orthogonal to the axis of the body section. The flange portion includes two major diameter forming portions that are symmetrically provided with the axis of the body section interposed therebetween to form the major diameter of the syringe assembly. The minor diameter is 10 to 13 mm and is equal to or larger than the outer diameter of the body section. The major diameter is 12 to 15 mm and is larger than the minor diameter.

According to the syringe assembly configured as described above, the total length is not too long and the outer diameter is not too large, and thus the size of a drug solution administration device using the syringe assembly can be reduced. In addition, since the syringe assembly includes the two major diameter forming portions protruding outward from the outer peripheral surface of the body section, the syringe assembly can be reliably held inside the drug solution administration device.

A width in a radial direction of the proximal surface may be 0.75 mm or more over an entire circumference.

With this configuration, during vacuum plugging, a contact surface (seal surface) between the proximal surface and a seal member for forming vacuum is increased, so that the vacuum plugging can be reliably performed.

The flange portion may be provided at a proximal end of the syringe assembly to form a part of the proximal surface.

With this configuration, since the flange portion forms a part of the proximal surface, when the syringe assembly is supported in the flange portion during vacuum plugging, a support surface and the proximal surface are close to each other, so that the vacuum plugging can be more reliably performed.

A thickness in an axial direction of the flange portion may be 1.0 to 2.5 mm.

With this configuration, since an increase in the thickness of the flange portion is suppressed, the size of the drug solution administration device can be further reduced.

The flange portion may include two minor diameter forming portions that are symmetrically provided with the axis of the body section interposed therebetween to form the minor diameter of the syringe assembly. The flange portion may be formed of the two major diameter forming portions and the two minor diameter forming portions to have an annular shape along a circumferential direction of the body section.

With this configuration, while the size of the drug solution administration device can be reduced, the flange portion can be stably supported during vacuum plugging.

The flange portion may include only two major diameter forming portions that are intermittently provided in a circumferential direction of the body section. The minor diameter may be equal to the outer diameter of the body section.

With this configuration, since the minor diameter of the syringe assembly is reduced, the size of the drug solution administration device can be further reduced.

The syringe assembly may further include a cap that seals the distal opening of the distal nozzle portion.

In addition, according to embodiments disclosed here, there is provided a prefilled syringe including: the above syringe assembly; the drug solution with which the lumen of the body section is filled; and a gasket that is disposed inside the body section to be slidable in the body section.

According to such a syringe assembly and prefilled syringe, the size of the drug solution administration device can be more effectively reduced.

### BRIEF DESCRIPTION OF THE DRAWINGS

FIG. 1 is a schematic configuration view of a drug solution administration device and an administration tool.

FIG. 2 is a perspective view illustrating a state of the drug solution administration device from which a housing is removed.

FIG. 3A is a side view of a syringe assembly according to a first embodiment. FIG. 3B is a view of the syringe assembly illustrated in FIG. 3A as seen from a proximal side.

FIG. 4 is a cross-sectional view along line IV-IV in FIG. 3A.

FIG. 5 is a perspective view of a syringe assembly according to a second embodiment.

FIG. 6A is a side view of the syringe assembly illustrated in FIG. 5. FIG. 6B is a view of the syringe assembly illustrated in FIG. 5 as seen from a proximal side.

FIG. 7 is a perspective view of a syringe assembly according to a third embodiment.

FIG. 8A is a side view of the syringe assembly illustrated in FIG. 7. FIG. 8B is a view of the syringe assembly illustrated in FIG. 7 as seen from a proximal side.

### DETAILED DESCRIPTION

Set forth below with reference to the accompanying drawings is a detailed description of embodiments of a syringe assembly and prefilled syringe representing examples of the inventive syringe assembly and prefilled syringe. Note that in second and third embodiments and modification examples thereof, the same or similar elements as a first embodiment are denoted by the same reference signs and detailed descriptions will be omitted.

### First Embodiment

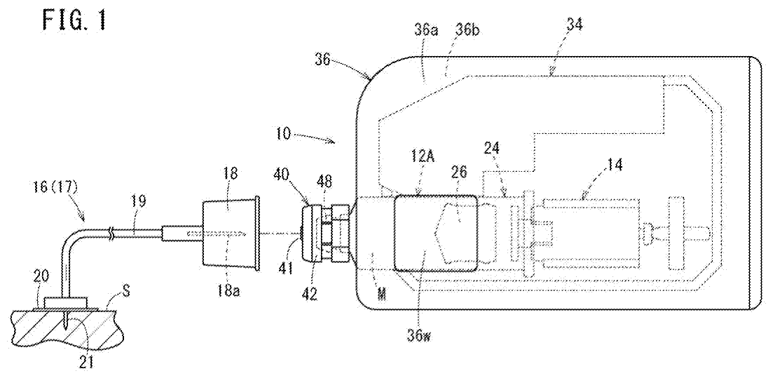

A drug solution administration device 10 according to the present embodiment illustrated in FIG. 1 is used to administer a drug solution M into a living body. The drug solution administration device 10 continuously administers the drug solution M, with which a syringe assembly 12A is filled, into the living body by a pressing operation of a plunger assembly 14 over a relatively long time (for example, approximately several minutes to several hours). The drug solution administration device 10 may intermittently administer the drug solution M into the living body. Examples of the drug solution M include protein preparations, narcotic analgesics, diuretics, and the like.

As illustrated in FIG. 1, during use of the drug solution administration device 10, for example, a patch-type tube/needle assembly 17 as an administration tool 16 is connected to the drug solution administration device 10, and the drug solution M discharged from the syringe assembly 12A is injected into the body of a patient via the tube/needle assembly 17. The tube/needle assembly 17 includes a connector 18 which can be connected to a distal nozzle portion 48 of the syringe assembly 12A, a liquid delivery tube 19 which is flexible and of which one end portion is connected to the connector 18, a patch portion 20 which is connected to the other end of the liquid delivery tube 19 to be attachable to skin S, and a puncture needle 21 which protrudes from the patch portion 20. The puncture needle 21 substantially perpendicularly punctures the skin S. Note that the puncture needle 21 may obliquely puncture the skin S.

Note that the administration tool 16 connected to the drug solution administration device 10 is not limited to the above patch-type tube/needle assembly 17 and, for example, a puncture needle (winged needle or the like) may be connected to a distal end of the liquid delivery tube 19. Alternatively, the administration tool 16 may be a bent needle that can be connected to the distal nozzle portion 48 of the syringe assembly 12A without via the liquid delivery tube 19. In this case, for example, the bent needle is bent by approximately 90° downward from the distal nozzle portion 48 of the syringe assembly 12A, and perpendicularly punctures the skin S as the drug solution administration device 10 is fixed (affixed) to the skin S. In addition, a form where the distal nozzle portion 48 of the syringe assembly 12A, the administration tool 16, and a part of the needle are located inside the syringe assembly 12A and a distal end of the needle protrudes from the syringe assembly 12A may be adopted. Also in this case, as the drug solution administration device 10 is fixed (affixed) to the skin S, the needle perpendicularly punctures the skin S.

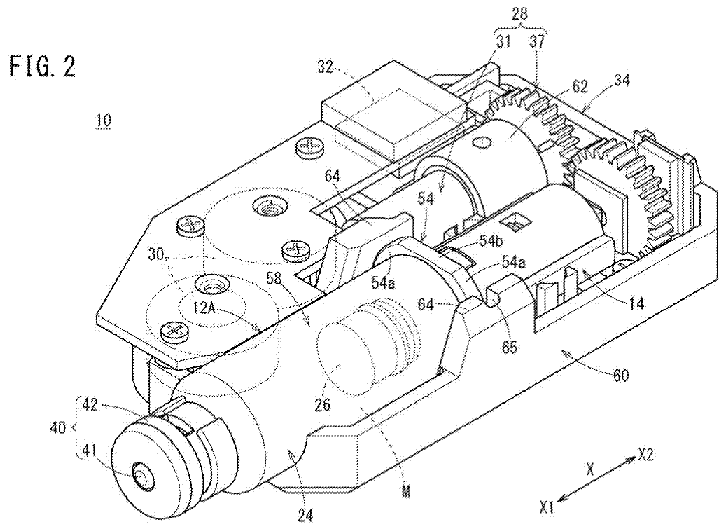

As illustrated in FIG. 1 or 2, the drug solution administration device 10 includes the syringe assembly 12A that includes a syringe 24 filled with the drug solution M, a gasket 26 that is slidably disposed inside the syringe 24, the plunger assembly 14 that can extend in an axial direction (arrow X direction) and can press the gasket 26 in a distal direction (arrow X1 direction), a drive mechanism 28 that drives the plunger assembly 14, a battery 30 that supplies electric power required to operate the drug solution administration device 10, a control unit 32 that controls the drive mechanism 28, a chassis structure 34 that supports the syringe assembly 12A, the plunger assembly 14, and the drive mechanism 28, and a housing 36 that accommodates the above components.

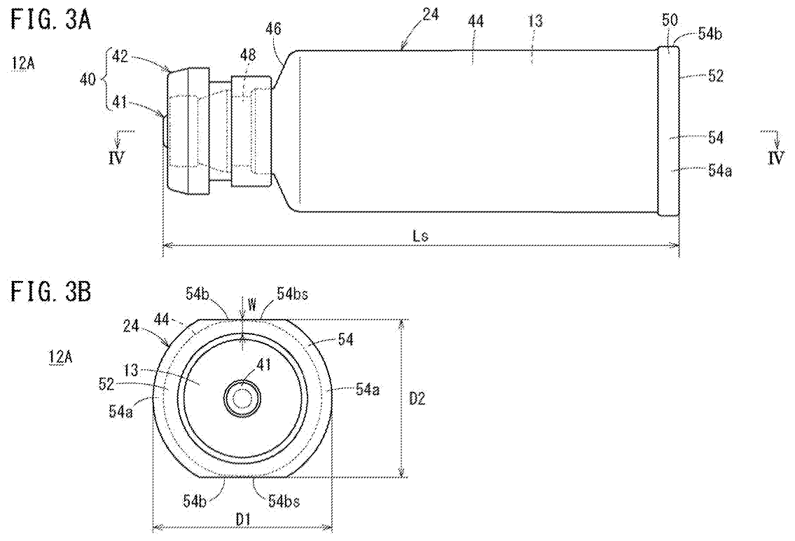

As illustrated in FIG. 2, the syringe assembly 12A includes the syringe 24 and a cap 40 mounted on the syringe 24. In FIG. 3A, a total length Ls (length from a distal surface of the cap 40 to a proximal surface 52 of the syringe 24) of the syringe assembly 12A is 30 to 40 mm.

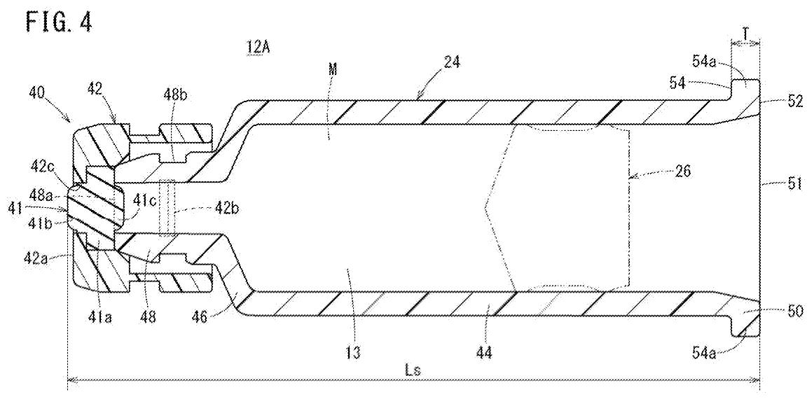

As illustrated in FIG. 4, the syringe 24 is formed in a hollow cylindrical shape. Specifically, the syringe 24 includes a body section 44 including a lumen 13, a shoulder section 46 that is reduced in diameter from a distal end of the body section 44, the distal nozzle portion 48 that protrudes from the shoulder section 46 in the distal direction, and a proximal portion 50 that is provided at a proximal end of the body section 44. The syringe 24 is prefilled with the drug solution M (FIGS. 1 and 2). The syringe 24 may be made of a transparent material.

The body section 44 includes the lumen 13 that is fillable with 0.3 to 1 mL of the drug solution M. The body section 44 has an inner diameter of 8.0 to 9.0 mm and an outer diameter of 10 to 12 mm. The ratio of the outer diameter of the body section 44 and the total length Ls of the syringe assembly 12A is 1:2.5 to 1:4. The distal nozzle portion 48 has a distal opening 48a (discharge port) that communicates with the lumen 13 of the body section 44.

The proximal portion 50 of the syringe 24 has a proximal opening 51 that communicates with the lumen 13 of the body section 44, the proximal surface 52 that encircles the proximal opening 51, and a flange portion 54 that protrudes outward from an outer peripheral surface of the body section 44. The proximal surface 52 is orthogonal to an axis of the body section 44 and is substantially flat. When the gasket 26 (FIG. 2) is inserted into the syringe 24 by a vacuum plugging method, a seal member for forming vacuum which is provided in a vacuum plugging apparatus is airtightly in contact with the entire circumference of the proximal surface 52 of the syringe 24.

As illustrated in FIG. 3B, the flange portion 54 protrudes outward from the outer peripheral surface of the body section 44 such that the syringe assembly 12A has a major diameter D1 and a minor diameter D2 in directions orthogonal to the axis of the body section 44. The minor diameter D2 is 10 to 13 mm and is equal to or larger than the outer diameter of the body section 44. The major diameter D1 is 12 to 15 mm and is larger than the minor diameter D2.

The flange portion 54 includes two major diameter forming portions 54a that are symmetrically provided with the axis of the body section 44 interposed therebetween to form the major diameter D1 of the syringe assembly 12A, and two minor diameter forming portions 54b that are symmetrically provided with the axis of the body section 44 interposed therebetween to form the minor diameter D2 of the syringe assembly 12A.

The two major diameter forming portions 54a protrude from the outer peripheral surface of the body section 44 in opposite directions, and are formed in an arc shape extending along a circumferential direction of the body section 44. The two minor diameter forming portions 54b protrude from the outer peripheral surface of the body section 44 in opposite directions. Outer surfaces 54bs (protruding end surfaces) of the two minor diameter forming portions 54b are substantially flat and are parallel to each other.

The flange portion 54 is formed of the two major diameter forming portions 54a and the two minor diameter forming portions 54b to have an annular shape along the circumferential direction of the body section 44. The flange portion 54 is provided at a proximal end of the syringe assembly 12A to form a part of the proximal surface 52 of the syringe 24. It is preferable that a width W (minimum width portion) in a radial direction of the proximal surface 52 is 0.75 mm or more. Namely, it is preferable that the width W in the radial direction of the proximal surface 52 is 0.75 mm or more over the entire circumference. The width of the proximal surface 52 in the major diameter forming portion 54a is larger than the width of the proximal surface 52 in the minor diameter forming portion 54b. The width W of the proximal surface 52 is minimum in a central portion of the minor diameter forming portion 54b.

In FIG. 4, a thickness T in the axial direction of the flange portion 54 is preferably 1.0 to 2.5 mm. Note that the flange portion 54 may be provided closer to a distal side than the proximal end of the syringe assembly 12A.

The cap 40 is made of an elastic resin material such as a rubber material or an elastomer material, and includes a seal member 41 that seals the distal opening 48a of the distal nozzle portion 48 and a cover member 42 that holds the seal member 41, has a tubular shape, and is harder than the seal member 41. The seal member 41 includes a proximal seal portion 41a forming a central portion in a thickness direction, a distal projection portion 41b that protrudes from a distal surface of the proximal seal portion 41a in the distal direction, and a proximal projection portion 41c that protrudes from a proximal surface of the proximal seal portion 41a in a proximal direction. The distal projection portion 41b protrudes slightly further in the distal direction than a distal surface 42a of the cover member 42.

Note that a distal surface of the distal projection portion 41b may be at the same axial position as the distal surface 42a of the cover member 42, or may be located closer to a proximal side than the distal surface 42a. In this case, the length from the distal surface 42a of the cover member 42 to the proximal surface 52 of the syringe 24 is the total length Ls of the syringe assembly 12A. When the connector 18 illustrated in FIG. 1 is connected to the distal nozzle portion 48, the seal member 41 is punctured by a needle 18a provided in the connector 18.

The cover member 42 has a through-hole 42c at a distal end thereof, and the distal projection portion 41b of the seal member 41 is inserted into the through-hole 42c. A distal surface of the seal member 41 is exposed from the through-hole 42c of the cover member 42. The seal member 41 is fixed to the distal nozzle portion 48 of the syringe assembly 12A by the cover member 42. A claw portion 42b provided in an inner peripheral portion of the cover member 42 is engaged with an engagement groove 48b provided in an annular shape in an outer peripheral portion of the distal nozzle portion 48, so that the cover member 42 is prevented from coming off from the distal nozzle portion 48.

In FIG. 2, the gasket 26 liquid-tightly closes a proximal side of the lumen 13 of the syringe 24. In an initial state of the drug solution administration device 10, the gasket 26 is located on the distal side of a proximal end of the syringe 24. An outer peripheral portion of the gasket 26 is liquid-tightly in close contact with an inner peripheral surface of the syringe 24 (body section 44). The syringe assembly 12A, the drug solution M, and the gasket 26 form a prefilled syringe 58.

The plunger assembly 14 is configured such that the gasket 26 advances in the syringe 24 to push the drug solution M out from the syringe assembly 12A. In the initial state of the drug solution administration device 10, a distal portion of the plunger assembly 14 is inserted into a proximal portion of the syringe 24. The drive mechanism 28 includes a motor 31 that has the battery 30 as a power source and is controlled to be driven by a control operation of the control unit 32, and a drive gear 37 that is fixed to an output shaft of the motor 31. Note that the distal portion of the plunger assembly 14 may be configured to be not inserted into the syringe 24 in the initial state but to be inserted into the syringe 24 when the drug solution M is to be pushed out.

The chassis structure 34 is disposed in the housing 36 (refer to FIG. 1). The syringe assembly 12A, the drive mechanism 28, and the plunger assembly 14 are fixed to predetermined positions of the chassis structure 34, respectively. The chassis structure 34 includes a chassis main body member 60 and a motor holding member 62 that is fixed to the chassis main body member 60 and holds the motor 31 between the chassis main body member 60 and the motor holding member 62.

The chassis main body member 60 includes a flange holding portion 64 that protrudes upward and holds the two major diameter forming portions 54a of the flange portion 54 of the syringe 24. The flange holding portion 64 is provided with a holding groove 65 into which one of the major diameter forming portions 54a is inserted. A movement in the axial direction of the syringe assembly 12A with respect to the chassis structure 34 is restricted by the flange holding portion 64.

In FIG. 1, the housing 36 is a hollow member configured to accommodate the syringe assembly 12A, the gasket 26, the plunger assembly 14, the drive mechanism 28, the battery 30, the control unit 32, and the chassis structure 34 described above. The distal nozzle portion 48 of the syringe assembly 12A protrudes from the housing 36, and the cap 40 is exposed outside. A window portion 36w made of a transparent material is provided in an upper surface 36a of the housing 36.

The drug solution administration device 10 can be configured as, for example, a patch type that is affixed to the skin S of the patient to be used. In such a patch type, an attachment portion (adhesion portion) that has a sheet shape and is attachable to the skin S is provided on a bottom surface 36b of the housing 36. In the initial state of the drug solution administration device 10, a peelable protective sheet is affixed to an attachment surface of the attachment portion.

Note that the drug solution administration device 10 may be configured as a type where a mounting tool such as a hook or a clip is provided on the bottom surface 36b of the housing 36 and is hooked on clothes (for example, waist portion of the pants) of the patient, so that the drug solution administration device 10 is attached.

Next, an operation of the drug solution administration device 10 including the syringe assembly 12A configured as described above will be described.

When the drug solution administration device 10 is to be used, the administration tool 16 is connected to the drug solution administration device 10. Specifically, the connector 18 is connected to the cap 40 of the syringe assembly 12A.

Then, the drug solution administration device 10 is affixed to the skin S of the patient or is mounted on the clothes to be attached to the patient. Next, the puncture needle 21 of the administration tool 16 punctures the skin S of the patient. Note that before the puncture needle 21 punctures the skin S, the drug solution administration device 10 may be attached to the patient.

Then, when the drug solution administration device 10 receives a predetermined operation start command, the plunger assembly 14 pushes the gasket 26 to cause the gasket 26 to advance in the syringe 24, so that the drug solution M in the syringe 24 is pushed out. The drug solution M pushed out from inside the syringe 24 is administered (injected) into the body of the patient via the administration tool 16 that has punctured the patient.

Note that the drug solution administration device 10 may be used in the following procedure instead of the above procedure.

The puncture needle 21 of the administration tool 16 punctures the skin S of the patient. Next, the administration tool 16 is connected to the drug solution administration device 10. Next, the drug solution administration device 10 is affixed to the skin S of the patient or is mounted on the clothes to be attached to the patient.

The syringe assembly 12A according to the first embodiment has the following effects.

As described above, the total length Ls of the syringe assembly 12A is 30 to 40 mm, the body section 44 has an inner diameter of 8.0 to 9.0 mm and an outer diameter of 10 to 12 mm, and the ratio of the outer diameter of the body section 44 and the total length Ls of the syringe assembly 12A is 1:2.5 to 1:4. The flange portion 54 includes the two major diameter forming portions 54a that are symmetrically provided with the axis of the body section 44 interposed therebetween to form the major diameter D1 of the syringe assembly 12A. The minor diameter D2 of the syringe assembly 12A is 10 to 13 mm and is equal to or larger than the outer diameter of the body section 44, and the major diameter D1 of the syringe assembly 12A is 12 to 15 mm and is larger than the minor diameter D2. In the syringe assembly 12A configured in such a manner, the total length Ls is not too long and the outer diameter is not too large, and thus the size of the drug solution administration device 10 using the syringe assembly 12A can be reduced. In addition, since the syringe assembly 12A includes the two major diameter forming portions 54a protruding outward from the outer peripheral surface of the body section 44, the syringe assembly 12A can be reliably held inside the drug solution administration device 10.

As illustrated in FIG. 3B, the flange portion 54 includes the two minor diameter forming portions 54b that are symmetrically provided with the axis of the body section 44 interposed therebetween to form the minor diameter D2 of the syringe assembly 12A, and the flange portion 54 is formed of the two major diameter forming portions 54a and the two minor diameter forming portions 54b to have an annular shape along the circumferential direction of the body section 44. With this configuration, while the size of the drug solution administration device 10 can be reduced, the flange portion 54 can be stably supported during vacuum plugging.

When the width W in the radial direction of the proximal surface 52 of the syringe assembly 12A is 0.75 mm or more over the entire circumference, during vacuum plugging, a contact surface (seal surface) between the proximal surface 52 and the seal member 41 for forming vacuum is increased, so that the vacuum plugging can be reliably performed.

The flange portion 54 is provided at the proximal end of the syringe assembly 12A to form a part of the proximal surface 52. With this configuration, since the flange portion 54 forms a part of the proximal surface 52, when the syringe assembly 12A is supported in the flange portion 54 during vacuum plugging, a support surface and the proximal surface 52 are close to each other, so that the vacuum plugging can be more reliably performed.

In FIG. 4, when the thickness T in the axial direction of the flange portion 54 is 1.0 to 2.5 mm, since an increase in the thickness of the flange portion 54 is suppressed, the size of the drug solution administration device 10 can be further reduced.

### Second Embodiment

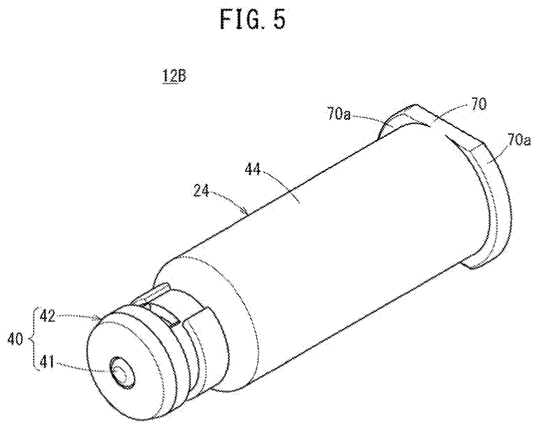

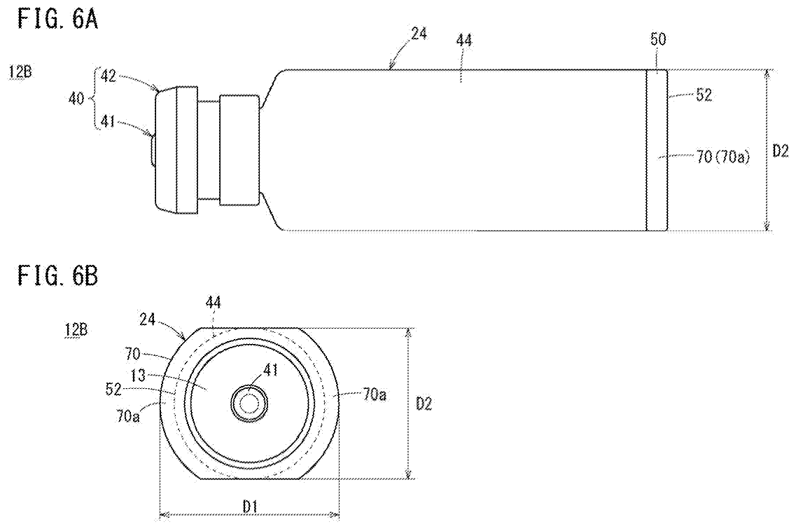

In a syringe assembly 12B according to a second embodiment illustrated in FIG. 5, a flange portion 70 protruding outward from the outer peripheral surface of the body section 44 includes only two major diameter forming portions 70a that are intermittently provided in the circumferential direction of the body section 44. As illustrated in FIGS. 6A and 6B, the minor diameter D2 of the syringe assembly 12B is equal to the outer diameter of the body section 44. Each of the major diameter forming portions 70a is provided in a range to half encircle the outer peripheral surface of the body section 44 in the circumferential direction. The length (length of the flange portion 70 in a direction perpendicular to the axis of the body section 44 and perpendicular to a protruding direction of the major diameter forming portion 70a) of each of the major diameter forming portions 70a along the radial direction of the body section 44 is equal to the minor diameter D2 of the syringe assembly 12B. Similar to the first embodiment, the flange portion 70 is provided at a proximal end of the syringe assembly 12B to form a part of the proximal surface 52 of the syringe assembly 12B. The drug solution administration device 10 (refer to FIG. 1) described above may adopt the syringe assembly 12B according to the second embodiment instead of the syringe assembly 12A according to the first embodiment.

According to the syringe assembly 12B according to the second embodiment, since the minor diameter D2 of the syringe assembly 12B is reduced by the extent that the flange portion 70 does not include the minor diameter forming portion, the size of the drug solution administration device 10 in which the syringe assembly 12B is used can be further reduced.

Note that in the second embodiment, portions common to the first embodiment can have the same or similar operations and effects as the first embodiment.

### Third Embodiment

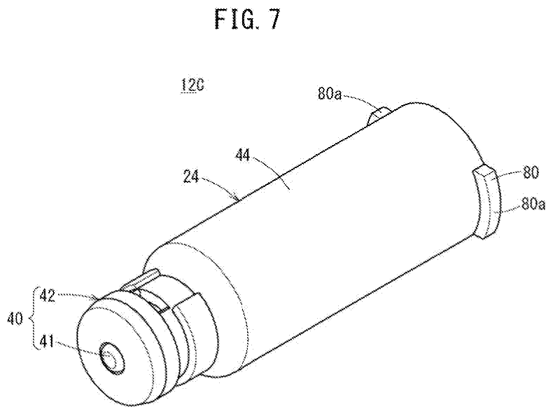

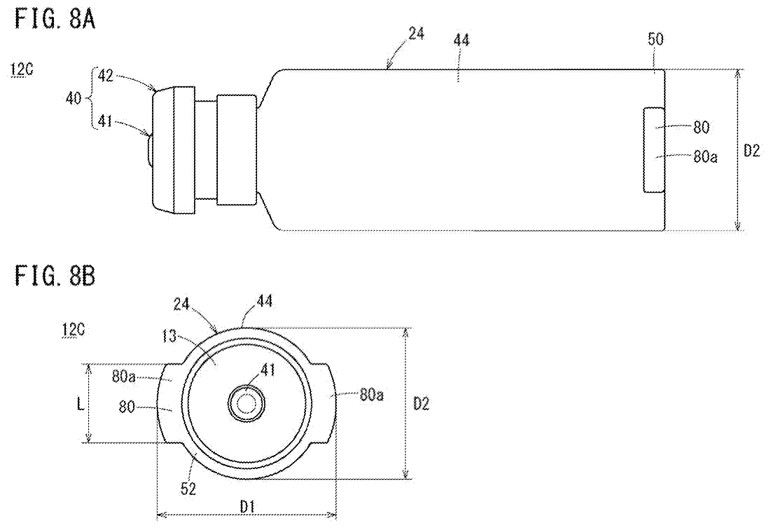

In a syringe assembly 12C according to a third embodiment illustrated in FIG. 7, a flange portion 80 protruding outward from the outer peripheral surface of the body section 44 includes only two major diameter forming portions 80a that are intermittently provided in the circumferential direction of the body section 44. As illustrated in FIGS. 8A and 8B, the minor diameter D2 of the syringe assembly 12C is equal to the outer diameter of the body section 44. Each of the major diameter forming portions 80a is provided in a range to less than half encircle the outer peripheral surface of the body section 44 in the circumferential direction. Therefore, the length L (length of the major diameter forming portion 80a in a direction perpendicular to the axis of the body section 44 and perpendicular to a protruding direction of the major diameter forming portion 80a) of each of the major diameter forming portions 80a along the radial direction of the body section 44 is equal to the minor diameter D2 of the syringe assembly 12C.

Similar to the first embodiment, the flange portion 80 is provided at a proximal end of the syringe assembly 12C to form a part of the proximal surface 52 of the syringe assembly 12C. The drug solution administration device 10 (refer to FIG. 1) described above may adopt the syringe assembly 12C according to the third embodiment instead of the syringe assembly 12A according to the first embodiment.

According to the syringe assembly 12C according to the third embodiment, the minor diameter D2 of the syringe assembly 12C is reduced by the extent that the flange portion 80 does not include the minor diameter forming portion. Moreover, a length L of the major diameter forming portion 80a is smaller than the minor diameter D2 of the syringe assembly 12C. For this reason, the size of the drug solution administration device 10 in which the syringe assembly 12C is used can be further reduced.

The detailed description above describes embodiments of a syringe assembly and prefilled syringe representing examples of the syringe assembly and prefilled syringe disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A syringe assembly comprising:
a body section including a lumen that is fillable with 0.3 to 1 mL of a drug solution;
a distal nozzle portion that is provided at a distal end of the body section and has a distal opening communicating with the lumen of the body section; and
a proximal portion that is provided at a proximal end of the body section, wherein
a total length of the syringe assembly is 30 to 40 mm,
the body section has an inner diameter of 8.0 to 9.0 mm and an outer diameter of 10 to 12 mm,
a ratio of the outer diameter of the body section and the total length of the syringe assembly is 1:2.5 to 1:4,
the proximal portion includes a proximal opening that communicates with the lumen of the body section, a proximal surface that encircles the proximal opening, is orthogonal to an axis of the body section, and is substantially flat, and a flange portion that protrudes outward from an outer peripheral surface of the body section such that the syringe assembly has a minor diameter and a major diameter in directions orthogonal to the axis of the body section,
the flange portion includes two major diameter forming portions that are symmetrically provided with the axis of the body section interposed therebetween to form the major diameter of the syringe assembly,
the minor diameter is 10 to 13 mm and is equal to or larger than the outer diameter of the body section, and
the major diameter is 12 to 15 mm and is larger than the minor diameter.

2. The syringe assembly according to claim 1,
wherein a width in a radial direction of the proximal surface is 0.75 mm or more over an entire circumference.

3. The syringe assembly according to claim 1,
wherein the flange portion is provided at a proximal end of the syringe assembly to form a part of the proximal surface.

4. The syringe assembly according to claim 1,
wherein a thickness in an axial direction of the flange portion is 1.0 to 2.5 mm.

5. The syringe assembly according to claim 1,
wherein the flange portion includes two minor diameter forming portions that are symmetrically provided with the axis of the body section interposed therebetween to form the minor diameter of the syringe assembly, and
the flange portion is formed of the two major diameter forming portions and the two minor diameter forming portions to have an annular shape along a circumferential direction of the body section.

6. The syringe assembly according to claim 1,
wherein the flange portion includes only two major diameter forming portions that are intermittently provided in a circumferential direction of the body section, and
the minor diameter is equal to the outer diameter of the body section.

7. The syringe assembly according to claim 1, further comprising:
a cap that seals the distal opening of the distal nozzle portion.

8. A prefilled syringe assembly comprising:
a body section including a lumen that is filled with 0.3 to 1 mL of a drug solution;
a distal nozzle portion that is provided at a distal end of the body section and has a distal opening communicating with the lumen of the body section;
a proximal portion that is provided at a proximal end of the body section; and
a gasket that is disposed inside the body section to be slidable in the body section, wherein
a total length of the syringe assembly is 30 to 40 mm,
the body section has an inner diameter of 8.0 to 9.0 mm and an outer diameter of 10 to 12 mm,
a ratio of the outer diameter of the body section and the total length of the syringe assembly is 1:2.5 to 1:4,
the proximal portion includes a proximal opening that communicates with the lumen of the body section, a proximal surface that encircles the proximal opening, is orthogonal to an axis of the body section, and is substantially flat, and a flange portion that protrudes outward from an outer peripheral surface of the body section such that the syringe assembly has a minor diameter and a major diameter in directions orthogonal to the axis of the body section,
the flange portion includes two major diameter forming portions that are symmetrically provided with the axis of the body section interposed therebetween to form the major diameter of the syringe assembly,
the minor diameter is 10 to 13 mm and is equal to or larger than the outer diameter of the body section, and
the major diameter is 12 to 15 mm and is larger than the minor diameter.

9. The prefilled syringe assembly according to claim 8,
wherein a width in a radial direction of the proximal surface is 0.75 mm or more over an entire circumference.

10. The prefilled syringe assembly according to claim 8,
wherein the flange portion is provided at a proximal end of the syringe assembly to form a part of the proximal surface.

11. The prefilled syringe assembly according to claim 8,
wherein a thickness in an axial direction of the flange portion is 1.0 to 2.5 mm.

12. The prefilled syringe assembly according to claim 8,
wherein the flange portion includes two minor diameter forming portions that are symmetrically provided with the axis of the body section interposed therebetween to form the minor diameter of the syringe assembly, and
the flange portion is formed of the two major diameter forming portions and the two minor diameter forming portions to have an annular shape along a circumferential direction of the body section.

13. The prefilled syringe assembly according to claim 8,
wherein the flange portion includes only two major diameter forming portions that are intermittently provided in a circumferential direction of the body section, and
the minor diameter is equal to the outer diameter of the body section.

14. The prefilled syringe assembly according to claim 8, further comprising:
a cap that seals the distal opening of the distal nozzle portion.

15. A method of administering drug solution, comprising:
a step of providing a prefilled syringe assembly, said prefilled syringe assembly comprising:
a body section including a lumen that is filled with 0.3 to 1 mL of the drug solution;
a distal nozzle portion that is provided at a distal end of the body section and has a distal opening communicating with the lumen of the body section;
a proximal portion that is provided at a proximal end of the body section; and
a gasket that is disposed inside the body section to be slidable in the body section, wherein
a total length of the syringe assembly is 30 to 40 mm,
the body section has an inner diameter of 8.0 to 9.0 mm and an outer diameter of 10 to 12 mm,
a ratio of the outer diameter of the body section and the total length of the syringe assembly is 1:2.5 to 1:4,
the proximal portion includes a proximal opening that communicates with the lumen of the body section, a proximal surface that encircles the proximal opening, is orthogonal to an axis of the body section, and is substantially flat, and a flange portion that protrudes outward from an outer peripheral surface of the body section such that the syringe assembly has a minor diameter and a major diameter in directions orthogonal to the axis of the body section,
the flange portion includes two major diameter forming portions that are symmetrically provided with the axis of the body section interposed therebetween to form the major diameter of the syringe assembly,
the minor diameter is 10 to 13 mm and is equal to or larger than the outer diameter of the body section, and
the major diameter is 12 to 15 mm and is larger than the minor diameter,
a step of puncturing skin of the patient with a needle;
a step of pushing the gasket toward the distal nozzle portion to cause the drug solution in the lumen of the body section to be administered to the patient through the needle.

16. The method according to claim 15,
wherein a width in a radial direction of the proximal surface is 0.75 mm or more over an entire circumference.

17. The method according to claim 15,
wherein the flange portion is provided at a proximal end of the syringe assembly to form a part of the proximal surface.

18. The method according to claim 15,
wherein a thickness in an axial direction of the flange portion is 1.0 to 2.5 mm.

19. The method according to claim 15,
wherein the flange portion includes two minor diameter forming portions that are symmetrically provided with the axis of the body section interposed therebetween to form the minor diameter of the syringe assembly, and
the flange portion is formed of the two major diameter forming portions and the two minor diameter forming portions to have an annular shape along a circumferential direction of the body section.

20. The method according to claim 15,
wherein the flange portion includes only two major diameter forming portions that are intermittently provided in a circumferential direction of the body section, and
the minor diameter is equal to the outer diameter of the body section.
